(19) United States
(12) Patent Application Publication
(10) Pub. No.: US 2024/0019352 A1
(43) Pub. Date: Jan. 18, 2024

(54) DEVICE, SENSOR, SENSING METHOD, SENSOR SYSTEM, AND POWER GENERATION METHOD

(71) Applicant: TRIPOD DESIGN CO., LTD., Tokyo (JP)

(72) Inventors: Satoshi NAKAGAWA, Tokyo (JP); Hirotaka OOI, Gunma (JP)

(73) Assignee: TRIPOD DESIGN CO., LTD., Tokyo (JP)

(21) Appl. No.: 18/032,910

(22) PCT Filed: Aug. 11, 2021
(86) PCT No.: PCT/JP2021/029687

§ 371 Date: Apr. 20, 2023

(30) Foreign Application Priority Data
Oct. 23, 2020 (JP) 2020-178432
Aug. 11, 2021 (JP) 2020178432

(51) Int. Cl. G01N 15/10; H02N 1/00

(52) U.S. Cl. CPC G01N 15/1031; H02N 1/008

(57) ABSTRACT

An object is to detect a trace substance and to generate energy by adsorption of the trace substance. A device includes a first electrode and a second electrode, wherein the first electrode and the second electrode are not electrically connected, a shortest distance between the first electrode and the second electrode is 0.001 μm or more and 100 μm or less, an absolute value of a difference between a standard electrode potential of the first electrode and a standard electrode potential of the second electrode is 0.1 V or more, and surfaces of the first electrode and the second electrode are partly or entirely exposed.

### TECHNICAL FIELD

The present invention relates to a device.

### BACKGROUND ART

Sensors that measure a concentration of a substance, such as a hygrometer, a dew point meter, a glucose sensor, and a pH sensor, measure the amount of substance attached between electrodes by measuring a current flowing between the electrodes using the fact that the resistance between the electrodes changes depending on the substance attached between the electrodes.

Patent Literature 1 discloses a sensor that includes a pair of comb-shaped electrodes and a reagent layer formed between the comb-shaped electrodes, applies a voltage between the electrodes, and measures a current between the electrodes to calculate a concentration of a target in a sample. However, in this configuration, in addition to a power supply for operating a current measurement circuit, a current flowing between the comb-shaped electrodes is required, and there is a problem that power consumption increases.

In addition, Patent Literature 2 discloses a humidity sensor that includes a pair of electrodes and a moisture-sensitive member whose physical quantity changes by adsorbing moisture between the electrodes, and measures humidity by converting a capacitance between the electrodes into a voltage. As a method for converting a capacitance into a voltage, it is disclosed to use a switched capacitor circuit. However, in this method, since it is required to allow a current to always flow between the electrodes and drive power of the switched capacitor circuit is required, there is a problem that power consumption increases. Furthermore, a need to use a complicated circuit is also a problem from the viewpoint of cost.

In addition, Patent Literature 3 discloses a comb-shaped electrode in which a first electrode and a second electrode are formed in a comb shape and a secondary battery using the same.

### CITATION LIST

### Patent Literature

Patent Literature 1: JP 6553554 B2
Patent Literature 2: JP 2008-268025 A
Patent Literature 3: WO 2014/038455 A

### SUMMARY OF INVENTION

### Technical Problem

An object of the present invention is to provide a device that generates electric power using fine particles.

### Solution to Problem

The present invention solves the above problem by any of the following [1] to [26].

[1] A device comprising a first electrode and a second electrode, wherein the first electrode and the second electrode are not electrically connected, a shortest distance between the first electrode and the second electrode is 0.001 μm or more and 100 μm or less, an absolute value of a difference between a standard electrode potential of the first electrode and a standard electrode potential of the second electrode is 0.1 V or more, and surfaces of the first electrode and the second electrode are partly or entirely exposed;

[2] The device according to [1], wherein the device includes a substrate, and the first electrode and the second electrode are physically connected via the substrate;

[3] The device according to [1] or [2], wherein the shortest distance between the first electrode and the second electrode is 10 μm or less;

[4] The device according to any one of [1] to [3], wherein the first electrode and the second electrode have a comb shape;

[5] The device according to any one of [2] to [4], wherein the first electrode and the second electrode are formed on a surface of the substrate;

[6] The device according to any one of [1] to [5], wherein the first electrode contains gold, silver, copper, platinum, or carbon;

[7] The device according to any one of [1] to [6], wherein the second electrode contains zinc, aluminum, magnesium, chromium, titanium, tin, iron, lithium, or sodium;

[8] The device according to any one of [1] to [7], wherein the first electrode and/or the second electrode has a laminated structure of a plurality of materials;

[9] The device according to any one of [2] to [8], wherein the first electrode and/or the second electrode is connected to the substrate via an adhesive layer;

[10] The device according to any one of [1] to [9], wherein an electrical resistance between the first electrode and the second electrode is 10 kΩ or more;

[11] The device according to any one of [2] to [10], wherein an electrical conductivity or ionic conductivity of the substrate is 1 S/cm or less;

[12] The device according to any one of [2] to [11], wherein a surface free energy of the substrate is 32 mJ/m² or more and 2,000 mJ/m² or less;

[13] The device according to any one of [2] to [12], wherein an amount of hydroxyl groups on a surface of the substrate is 0.1 atomic % or more and 90 atomic % or less;

[14] The device according to any one of [2] to [11], wherein a surface free energy of the substrate is 10 mJ/m² or more and 2,000 mJ/m² or less;

[15] The device according to any one of [2] to and [14], wherein a water-repellent material is formed on a surface of the substrate;

[16] The device according to any one of [2] to [15], wherein a contact angle of water to a surface of the substrate is 90° or less;

[17] The device according to any one of [2] to [16], wherein an arithmetic average roughness Ra of a surface of the substrate is 0.001 μm or more and 1 μm or less;

[18] The device according to any one of [2] to [17], wherein the substrate is glass, polyimide, PET, PEN, or a silicon wafer;

[19] A sensor comprising a first electrode and a second electrode, wherein the first electrode and the second electrode are not electrically connected, a shortest distance between the first electrode and the second electrode is 0.001 μm or more and 100 μm or less, and an absolute value of a difference between a standard electrode potential of the first electrode and a standard electrode potential of the second electrode is 0.1 V or more;

[20] The sensor according to [19], further comprising a third electrode, wherein the first electrode and the third electrode are not electrically connected, the second electrode and the third electrode are not electrically connected, a shortest distance between the first electrode or the second electrode and the third electrode is 0.001 μm or more and 100 μm or less, and an absolute value of a difference between a standard electrode potential of the first electrode or the second electrode and a standard electrode potential of the third electrode is 0.1 V or more;

[21] A sensing method comprising detecting that a particle having a size of 100 μm or less and containing an ionized molecule comes into contact with the first electrode and the second electrode, in a device including the first electrode and the second electrode, in which the first electrode and the second electrode are not electrically connected, a shortest distance between the first electrode and the second electrode is 0.001 μm or more and 100 μm or less, and an absolute value of a difference between a standard electrode potential of the first electrode and a standard electrode potential of the second electrode is 0.1 V or more;

[22] The sensing method according to [21], wherein the size of the particle is 10 μm or less;

[23] A sensor system comprising a first sensor and a second sensor, wherein the first sensor includes a first electrode and a second electrode, the first electrode and the second electrode are not electrically connected, a shortest distance between the first electrode and the second electrode is 0.001 μm or more and 100 μm or less, an absolute value of a difference between a standard electrode potential of the first electrode and a standard electrode potential of the second electrode is 0.1 V or more, the second sensor includes a third electrode and a fourth electrode, the third electrode and the fourth electrode are not electrically connected, a shortest distance between the third electrode and the fourth electrode is 0.001 μm or more and 100 μm or less, an absolute value of a difference between a standard electrode potential of the third electrode and a standard electrode potential of the fourth electrode is 0.1 V or more, and the shortest distance between the first electrode and the second electrode is different from the shortest distance between the third electrode and the fourth electrode;

[24] A sensor system comprising a first sensor and a second sensor, wherein the first sensor includes a first electrode and a second electrode, the first electrode and the second electrode are not electrically connected, a shortest distance between the first electrode and the second electrode is 0.001 μm or more and 100 μm or less, an absolute value of a difference between a standard electrode potential of the first electrode and a standard electrode potential of the second electrode is 0.1 V or more, the second sensor includes a third electrode and a fourth electrode, the third electrode and the fourth electrode are not electrically connected, a shortest distance between the third electrode and the fourth electrode is 0.001 μm or more and 100 μm or less, an absolute value of a difference between a standard electrode potential of the third electrode and a standard electrode potential of the fourth electrode is 0.1 V or more, and a combination of a material constituting the first electrode and a material constituting the second electrode is different from a combination of a material constituting the third electrode and a material constituting the fourth electrode;

[25] A power generation method comprising generating power by bringing a particle having a size of 100 μm or less and containing an ionized molecule into contact with a first electrode and a second electrode, in a device including the first electrode and the second electrode, in which the first electrode and the second electrode are not electrically connected, the shortest distance between the first electrode and the second electrode is 0.001 μm or more and 100 μm or less, and an absolute value of a difference between a standard electrode potential of the first electrode and a standard electrode potential of the second electrode is 0.1 V or more;

The power generation method according to [25], wherein the size of the particle is 10 μm or less.

### BRIEF DESCRIPTION OF DRAWINGS

FIG. 1 is a view illustrating an example of an arrangement of electrodes according to an embodiment of the present invention.

FIG. 2 is a view illustrating an example of an arrangement of electrodes according to an embodiment of the present invention.

FIG. 3 is a view illustrating an example of a structure of an electrode according to an embodiment of the present invention.

FIG. 4 is a view for explaining an arrangement of electrodes in an Example according to an embodiment of the present invention.

### DESCRIPTION OF EMBODIMENTS

Hereinafter, embodiments of the present invention (hereinafter, referred to as “the present embodiments”) will be described in detail. Note that the present invention is not limited to the following embodiments, and various modifications can be made within the scope of the gist of the present invention.

(Device Configuration)

A device of the present embodiment includes a first electrode and a second electrode, the first electrode and the second electrode are not electrically connected, the shortest distance between the first electrode and the second electrode is 0.001 μm or more and 100 μm or less, an absolute value of a difference between a standard electrode potential of the first electrode and a standard electrode potential of the second electrode is 0.1 V or more, and surfaces of the first electrode and the second electrode are partly or entirely exposed.

In general, even when a substance is adsorbed between electrically insulated electrodes, no potential difference occurs between the electrodes. On the other hand, the inventors have found that when the distance between the electrodes is extremely reduced, a chemical reaction occurs between the electrodes due to adsorption of the substance, and a potential difference occurs.

(Device Including n Electrodes)

The device described above may include n electrodes. Here, n is an integer of 3 or more. The n electrodes from a first electrode and an n-th electrode are not electrically connected to each other. The shortest distance between a k-th electrode and at least one other electrode is 0.001 μm or more and 100 μm or less. Here, k is an integer of 1 or more and n or less. In addition, an absolute value of a difference between a standard electrode potential of the k-th electrode and a standard electrode potential of the at least one other electrode is 0.1 V or more. Surfaces of the n electrodes from the first electrode to the n-th electrode are partly or entirely exposed.

Among the n electrodes from the first electrode to the n-th electrode, two or more and (n−1) or fewer electrodes may have the same shape, material, and/or physical properties.

Among the n electrodes from the first electrode to the n-th electrode, three or more electrodes may be formed of different materials. Different pieces of information can be obtained from electrodes formed of different materials. Therefore, the device including the n electrodes preferably includes three or more electrodes formed of different materials. For example, in a case of a device including a first electrode, a second electrode, and a third electrode, in which a material of the first electrode, a material of the second electrode, and a material of the third electrode are different from each other, since a response to the adsorbed substance differs between a set of the first electrode and the second electrode and a set of the first electrode and the third electrode, a plurality of pieces of information on the characteristics of the adsorbed substance can be obtained.

The shortest distance between the k-th electrode and a j-th electrode may be constant or different for any k and j. Here, j is an integer of 1 or more and n or less, which is different from that of k. Different pieces of information can be obtained from sets of electrodes having the shortest distances between the electrodes that are different from each other. Therefore, the device including the n electrodes preferably includes a plurality of sets of electrodes having the shortest distances between the electrodes that are different from each other. For example, in a case of a device including a first electrode, a second electrode, and a third electrode, in which values of the shortest distance between the first electrode and the second electrode and the shortest distance between the first electrode and the third electrode are different from each other, since a size of a substance that can be detected differs between a set of the first electrode and the second electrode and a set of the first electrode and the third electrode, information on the size of the adsorbed substance can be obtained.

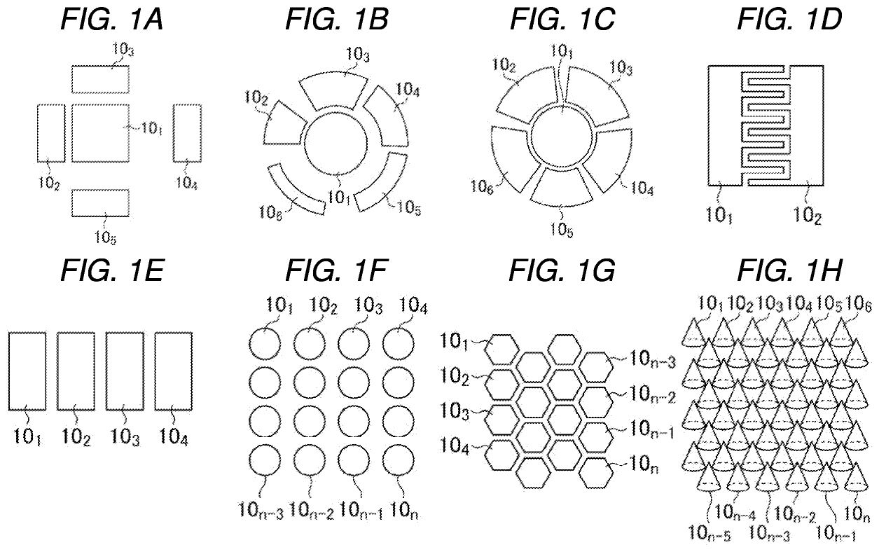

FIG. 1 is a view illustrating an example of an arrangement of electrodes according to an embodiment of the present invention. FIG. 1 illustrates an example of an arrangement of electrodes in a case where a plurality of electrodes are two-dimensionally arranged.

FIG. 1A is a view illustrating an example of an arrangement of electrodes of a device including five electrodes from a first electrode 10₁ to a fifth electrode 10₅. In FIG. 1a, each electrode has a plate-like quadrangular shape. The respective electrodes are arranged so that surfaces having large surface areas of the respective electrodes are positioned on the same plane. In addition, four electrodes from the second electrode 10₂ to the fifth electrode 10₅ are arranged concentrically around the first electrode 10₁. Distances between the electrodes 10 arranged at the closest distance, that is, adjacent electrodes, are different from each other.

FIG. 1B is a view illustrating an example of an arrangement of electrodes of a device including six electrodes from a first electrode 10₁ to a sixth electrode 10₆. In FIG. 1B, the first electrode 10₁ has a plate-like circular shape, and five electrodes from the second electrode 10₂ to the sixth electrode 10₆ have a plate-like shape surrounded by two arcs. The respective electrodes are arranged so that surfaces having large surface areas of the respective electrodes are positioned on the same plane. In addition, five electrodes from the second electrode 10₂ to the sixth electrode 10₆ are arranged concentrically around the first electrode 10₁. Distances between adjacent electrodes are different from each other.

FIG. 1C is a view illustrating an example of an arrangement of electrodes of a device including six electrodes from a first electrode 10₁ to a sixth electrode 10₆. In FIG. 1C, the first electrode 10₁ has a plate-like circular shape, and five electrodes from the second electrode 10₂ to the sixth electrode 10₆ have a plate-like shape surrounded by two arcs. The respective electrodes are arranged so that surfaces having large surface areas of the respective electrodes are positioned on the same plane. In addition, five electrodes from the second electrode 10₂ to the sixth electrode 10₆ are arranged concentrically around the first electrode 10₁. In addition, distances between the respective electrodes of the first electrode 10₁, the second electrode 10₂, the third electrode 10₃, the fourth electrode 10₄, the fifth electrode 10₅, and the sixth electrode 10₆ are equal to each other. With such an arrangement of the electrodes, the number of electrode pairs having the same distance between the electrodes can be easily increased.

FIG. 1D is a view illustrating an example of an arrangement of electrodes of a device including two electrodes of a first electrode 10₁ and a second electrode 10₂. In FIG. 1D, the first electrode 10₁ and the second electrode 10₂ have a plate-like comb shape. The first electrode 10₁ and the second electrode 10₂ are arranged so that surfaces having large surface areas are positioned on the same plane and comb teeth are engaged with each other. Distances between the electrodes of convex portions of the comb teeth of the first electrode 10₁ and the second electrode 10₂ may be equal to or different from each other. In addition, distances between the electrodes of convex portions and concave portions of the comb teeth of the first electrode 10₁ and the second electrode 10₂ may be equal to or different from each other. Furthermore, the distances between the electrodes of the convex portions of the comb teeth and the distances between the electrodes of the convex portions and the concave portions of the comb teeth of the first electrode 10₁ and the second electrode 10₂ may be equal to or different from each other. With such an arrangement of the electrodes, a length and a surface area of a portion where the electrodes face to each other can be increased, and thus sensitivity of a sensor described below can be increased. The number of convex portions of the comb teeth may be one or more. The number of convex portions of the comb teeth is preferably 10 or more and more preferably 100 or more. In addition, the number of convex portions of the comb teeth is preferably 10,000 or less, more preferably 1,000 or less, and still more preferably 500 or less. When the number of convex portions of the comb teeth is within the above range, it is possible to increase the sensitivity of the device as a sensor and improve a yield at the time of processing.

FIG. 1E is a view illustrating an example of an arrangement of electrodes of a device including four electrodes from a first electrode 10₁ to a fourth electrode 10₄. In FIG. 1E, each electrode has a plate-like quadrangular shape. The respective electrodes are arranged in a row in a horizontal direction, that is, in a one-dimensional array shape, so that surfaces having large surface areas are positioned on the same plane. In addition, distances between adjacent electrodes are equal to each other.

FIG. 1F is a view illustrating an example of an arrangement of electrodes of a device including n electrodes from a first electrode 10₁ to an n-th electrode 10ₙ. In FIG. 1F, each electrode has a plate-like circular shape. The respective electrodes are regularly arranged in a vertical direction and a horizontal direction so that surfaces having large surface areas are positioned on the same plane. That is, the respective electrodes are arranged in a two-dimensional array shape. In addition, distances between adjacent electrodes are equal to each other. With such an arrangement of the electrodes, a large number of electrodes can be formed in a small area.

FIG. 1G is a view illustrating an example of an arrangement of electrodes of a device including n electrodes from a first electrode 10₁ to an n-th electrode 10ₙ. In FIG. 1G, each electrode has a plate-like regular hexagonal shape. The respective electrodes are arranged so that surfaces having large surface areas are positioned on the same plane and gaps are uniform in a vertical direction and a horizontal direction. That is, the respective electrodes are arranged to form a honeycomb structure in a two-dimensional array shape. In addition, distances between adjacent electrodes are equal to each other. With such an arrangement, a distance between any electrode and an electrode adjacent thereto can be set to be constant. In a case where the shape of the electrode is an equilateral triangular shape, a square shape, or the like, the same arrangement can be used.

FIG. 1H is a view illustrating an example of an arrangement of electrodes of a device including n electrodes from a first electrode 10₁ to an n-th electrode 10ₙ. In FIG. 1H, each electrode has a cone shape. The respective electrodes are regularly arranged in a vertical direction and a horizontal direction so that bottom surfaces of the cones are positioned on the same plane. That is, the respective electrodes are arranged in a two-dimensional array shape. In addition, distances between adjacent electrodes are equal to each other.

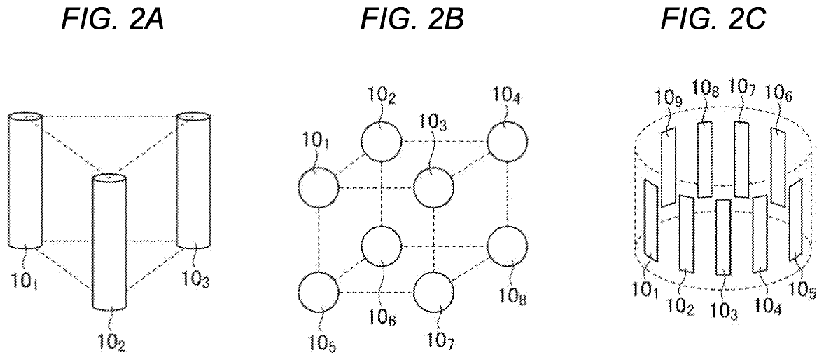

FIG. 2 is a view illustrating an example of an arrangement of electrodes according to an embodiment of the present invention. FIG. 2 illustrates an example of an arrangement of electrodes in a case where a plurality of electrodes are three-dimensionally arranged.

FIG. 2A is a view illustrating an example of an arrangement of electrodes of a device including three electrodes of a first electrode 10₁, a second electrode 10₂, and a third electrode 10₃. In FIG. 2A, each electrode has a cylindrical shape. The respective electrodes are arranged along a side of a triangular prism in a height direction. In a case where the respective electrodes are arranged along a side of an equilateral triangular prism in a height direction, distances between adjacent electrodes are equal to each other.

FIG. 2B is a view illustrating an example of an arrangement of electrodes of a device including eight electrodes from a first electrode 10₁ to an eighth electrode 10₈. In FIG. 2B, each electrode has a spherical shape. Each electrode is arranged at a position of each vertex of a rectangular parallelepiped. Distances between adjacent electrodes are equal to each other.

FIG. 2C is a view illustrating an example of an arrangement of electrodes of a device including nine electrodes from a first electrode 10₁ to a ninth electrode 10₉. In FIG. 2C, each electrode has a plate-like quadrangular shape. The respective electrodes are arranged in a row so that surfaces having large surface areas are arranged on a side surface of a cylinder and long sides of the quadrangles of the respective electrodes are parallel to a height direction of the cylinder. Distances between adjacent electrodes are equal to each other.

In addition to the examples illustrated in FIGS. 1 and 2, the device can have various electrode arrangements. For example, an electrode can be arranged at each vertex of a lattice such as a body-centered cubic lattice, a face-centered cubic lattice, or a hexagonal close-packed structure.

(Device Including Three Electrodes)

A first electrode 10₁ and a second electrode 10₂, and further a third electrode 10₃ are included, the first electrode 10₁ and the second electrode 10₂ are not electrically connected, the first electrode 10₁ and the third electrode 10₃ are not electrically connected, the second electrode 10₂ and the third electrode 10₃ are not electrically connected, the shortest distance between the first electrode 10₁ and the second electrode 10₂ is 0.001 μm or more and 100 μm or less, and an absolute value of a difference between a standard electrode potential of the first electrode 10₁ and a standard electrode potential of the second electrode 10₂ is 0.1 V or more.

The shortest distance between the third electrode 10₃ and the first electrode 10₁ and the shortest distance between the third electrode 10₃ and the second electrode 10₂ are not particularly limited, but one of them is preferably 0.001 μm or more and 100 μm or less. In addition, both the shortest distance between the third electrode 10₃ and the first electrode 10₁ and the shortest distance between the third electrode 10₃ and the second electrode 10₂ may be 0.001 μm or more and 100 μm or less.

An absolute value of a difference between a standard electrode potential of the third electrode 10₃ and the standard electrode potential of the first electrode 10₁ and an absolute value of a difference between the standard electrode potential of the third electrode 10₃ and the standard electrode potential of the second electrode 10₂ are not particularly limited, but one of them is preferably 0.1 V or more. In addition, both the absolute value of the difference between the standard electrode potential of the third electrode 10₃ and the standard electrode potential of the first electrode 10₁ and the absolute value of the difference between the standard electrode potential of the third electrode 10₃ and the standard electrode potential of the second electrode 10₂ may be 0.1 V or more.

A device including three electrodes 10 may have a structure in which the electrodes 10 are positioned along sides of a triangular prism in a height direction as illustrated in FIG. 2A. In the case of the structure in which the electrodes 10 are positioned on the sides of the triangular prism in the height direction, the shortest distance between the first electrode 10₁ and the second electrode 10₂, the shortest distance between the first electrode 10₁ and the third electrode 10₃, and the shortest distance between the second electrode 10₂ and the third electrode 10₃ are equal to each other.

(Common Electrode)

The shape of the electrode 10 is not particularly specified, but may have a one-dimensional, two-dimensional, or three-dimensional shape. The one-dimensional shape refers to a linear shape. The two-dimensional shape refers to a quadratic curve (for example, an ellipse, a parabola, a hyperbola, or the like) on a plane, a shape represented by a combination of a quadratic curve and a straight line, a planar shape (a polygon, an ellipse, a circle, a fan, or the like), a shape formed by a combination thereof, or the like. In particular, a comb shape and a honeycomb shape are preferable, and the comb shape is more preferable because the sensitivity of the sensor can be increased. The three-dimensional shape refers to a quadratic curve in a three-dimensional Euclidean space, a shape represented by a combination of a quadratic curve and a straight line, a curved surface shape, a three-dimensional shape (for example, a polyhedron, a cone, a twin cone, a frustum, a column, an ellipse, or the like), a shape formed by a combination thereof, or the like. In addition, the shapes of the first electrode 10₁ and the second electrode 10₂ may have regularity or may be irregular. Examples of the regular structure include a fractal structure. In addition, each electrode may have a biomimetic shape.

The absolute value of the difference between the standard electrode potentials of the first electrode 10₁ and the second electrode 10₂ is preferably 0.1 V or more, more preferably 0.2 V or more, still more preferably 0.5 V or more, and particularly preferably 1.0 V or more in order to measure trace components with high sensitivity. When the absolute value of the difference between the standard electrode potentials of the first electrode 10₁ and the second electrode 10₂ is 0.1 V or more, the device can be used as a sensor, and when the absolute value is 0.2 V or more, the device can be used as a power supply in combination with a boosting circuit. In addition, when the absolute value of the difference between the standard electrode potentials of the first electrode 10₁ and the second electrode 10₂ is 0.5 V or more, the measurement can be performed with a general-purpose measuring circuit, and when the absolute value is 1.0 V or more, trace components can be measured with high sensitivity. The absolute value of the difference between the standard electrode potentials of the first electrode 10₁ and the second electrode 10₂ is preferably 5.0 V or less, more preferably 2.5 V or less, and still more preferably 2.0 V or less.

In a device including n electrodes 10, for any k, an absolute value of a difference between a standard electrode potential of a k-th electrode 10ₖ and a standard electrode potential of at least one other electrode 10 satisfies the above conditions.

The first electrode 10₁ and the second electrode 10₂ are not electrically connected. The shortest distance between the first electrode 10₁ and the second electrode 10₂ is preferably 0.001 μm or more, more preferably 0.01 μm or more, and still more preferably 0.1 μm or more. When the shortest distance between the first electrode 10₁ and the second electrode 10₂ is 0.01 μm or more, the yield is improved, and when the shortest distance is 0.1 μm or more, insulation can be reliably performed.

In addition, the shortest distance between the first electrode 10₁ and the second electrode 10₂ is preferably 100 μm or less, more preferably 80 μm or less, still more preferably 30 μm or less, and particularly preferably 10 μm or less. When the shortest distance between the first electrode 10₁ and the second electrode 10₂ is 100 μm or less, droplets can be detected, when the shortest distance is 80 μm or less, an atomized substance can be detected, when the shortest distance is 30 μm or less, exhalation can be detected, and when the shortest distance is 10 μm or less, high-humidity air can be detected.

Here, the shortest distance between the first electrode 10₁ and the second electrode 10₂ refers to the minimum length among the lengths of line segments connecting any point on the first electrode 10₁ and any point on the second electrode 10₂.

In the device including the n electrodes 10, for any k, the shortest distance between the k-th electrode 10ₖ and the at least one other electrode 10 satisfies the above conditions.

(Material of Electrode)

A material of the electrode 10 may be a single material or a composite of a plurality of materials. The material of the electrode 10 may be, for example, a current collector, an active material, a binder, a conductive auxiliary agent, an electrolyte, a solvent, an additive, or the like.

(Structure of Electrode)

A structure of the electrode 10 is not particularly specified, but may be configured by a single layer or may include a laminated structure of a plurality of materials. Since the resistance of the electrode 10 can be reduced, it is preferable to have a laminated structure in which a layer of an active material, that is, an active material layer, is formed on a surface of a conductive layer. As the conductive layer, a current collector can be used. The structure of the electrode 10 may be a structure in which an active material is supported on a porous conductive layer, since it is easily manufactured.

In addition, the first electrode 10₁ and/or the second electrode 10₂ is preferably connected to a substrate described below via an adhesive layer. In this case, a conductive layer of the first electrode 10₁ and/or the second electrode 10₂ is preferably connected to the substrate described below via the adhesive layer. Since the first electrode 10₁ and/or the second electrode 10₂ is connected to the substrate via the adhesive layer, adhesion of the first electrode 10₁ and/or the second electrode 10₂ and the substrate can be improved. As the adhesive layer, a pressure-sensitive adhesive or an adhesive may be used. In addition, a metal such as titanium or chromium may be used.

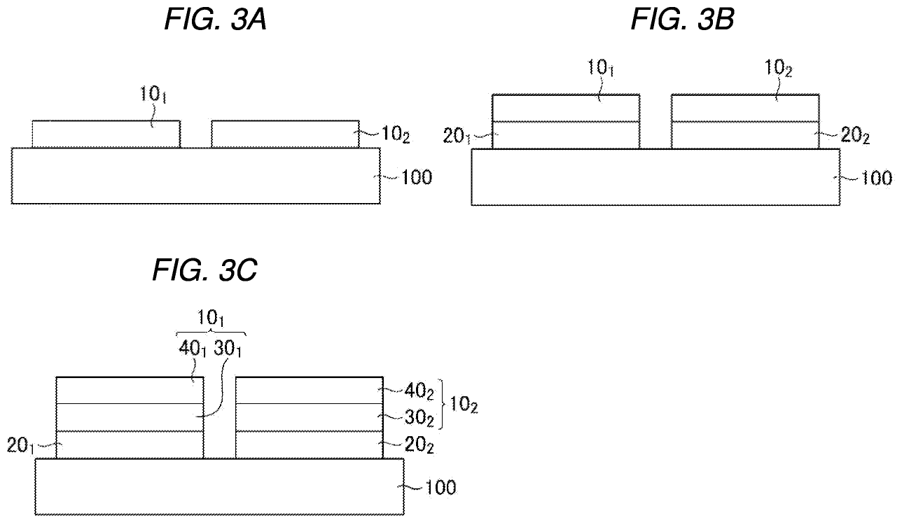

FIG. 3 is a view illustrating an example of a structure of an electrode according to an embodiment of the present invention. In FIG. 3, a first electrode 10₁ and a second electrode 10₂ are formed on a surface of a substrate.

FIG. 3A is a view illustrating an example of a structure of an electrode in which a first electrode 10₁ and a second electrode 10₂ are formed on a surface of a substrate 100. The first electrode 10₁ and the second electrode 10₂ are formed on one surface of the substrate 100.

FIG. 3B is a view illustrating an example of a structure of an electrode in which a first electrode 10₁ and a second electrode 10₂ are formed on a surface of a substrate 100 via adhesive layers 20, respectively. The first electrode 10₁ and the second electrode 10₂ are connected to the substrate 100 via an adhesive layer 20₁ and an adhesive layer 20₂, respectively. In addition, the first electrode 10₁ and the second electrode 10₂ are formed on one surface of the substrate 100.

FIG. 3C is a view illustrating an example of a structure of an electrode in which a first electrode 10₁ and a second electrode 10₂ that have a laminated structure are formed on a surface of a substrate 100 via adhesive layers 20, respectively. The first electrode 10₁ has a laminated structure in which an active material layer 40₁ is formed on a surface of a conductive layer 30₁. The active material layer 40₁ is formed on a surface of the conductive layer 30₁ opposite to the adhesive layer 20₁. In addition, the second electrode 10₂ also has a laminated structure similar to that of the first electrode 10₁. The conductive layer 30₁ of the first electrode 10₁ and a conductive layer 30₂ of the second electrode 10₂ are connected to the substrate 100 via the adhesive layer 20₁ and the adhesive layer 20₂, respectively. In addition, the first electrode 10₁ and the second electrode 10₂ are formed on one surface of the substrate 100.

(Current Collector)

Since a current collector has a role of conducting carriers generated by an oxidation-reduction reaction of the active material of the electrode, it is preferable to use a material having a low electrical resistance. The electrical resistance of the current collector is preferably 10 mΩcm or less, more preferably 1 mΩcm or less, and still more preferably 100 μΩcm or less.

The electrical resistance of the current collector is preferably 1 kΩ or less, more preferably 100Ω or less, and still more preferably 10Ω or less.

A material of the current collector is not particularly limited as long as it has conductivity, and for example, a carbon-based material, a metal material, conductive ceramic, a conductive plastic, or the like can be used.

Examples of the carbon-based material include activated carbon, carbon black (Ketjen black, acetylene black, channel black, furnace black, lamp black, thermal black, or the like), graphite, carbon nanotube, carbon nanohorn, graphene, and fullerene. As the metal material, metals such as gold, silver, copper, nickel, aluminum, titanium, vanadium, chromium, manganese, iron, cobalt, zinc, niobium, molybdenum, palladium, cadmium, indium, tin, antimony, lanthanum, tantalum, tungsten, platinum, and lead, and an oxide, a nitride, a carbide, a salt, and an alloy of these metals can be used.

Examples of the conductive ceramic include indium-tin oxide, indium-zinc oxide, and indium-gallium-zinc oxide.

As a conductive organic substance, a material formed of a n-conjugated molecule such as polythiophene, polyaniline, polypyrrole, polyacetylene, polyphenylene vinylene, or PEDOT, and a material formed of a n-conjugated molecule and a dopant such as PEDOT/PSS, a charge transfer complex such as TTF-TCNQ, and the like can be used.

The current collector is preferably a stable material that does not oxidize or reduce itself in the environment of use or a material that forms a stable film, and particularly preferably a carbon-based material, copper, stainless steel, aluminum, nickel, titanium, and an alloy thereof.

(Conductive Auxiliary Agent)

As the conductive auxiliary agent, a powder material having conductivity can be used.

A material of the conductive auxiliary agent is not particularly limited as long as it has conductivity, and for example, a carbon-based material, a metal material, a conductive ceramic, a conductive plastic, or the like can be used.

Examples of the carbon-based material include activated carbon, carbon black (Ketjen black, acetylene black, channel black, furnace black, lamp black, thermal black, or the like), graphite, carbon nanotube, carbon nanohorn, graphene, and fullerene.

As the metal material, metals such as gold, silver, copper, nickel, aluminum, titanium, vanadium, chromium, manganese, iron, cobalt, zinc, niobium, molybdenum, palladium, cadmium, indium, tin, antimony, lanthanum, tantalum, tungsten, platinum, and lead, and an oxide, a nitride, a carbide, a salt, and an alloy of these metals can be used.

Examples of the conductive ceramic include indium-tin oxide, indium-zinc oxide, and indium-gallium-zinc oxide. As a conductive organic substance, a material formed of a n-conjugated molecule such as polythiophene, polyaniline, polypyrrole, polyacetylene, polyphenylene vinylene, or PEDOT, and a material formed of a n-conjugated molecule and a dopant such as PEDOT/PSS, and the like can be used.

The conductive auxiliary agent is preferably a stable material that does not oxidize or reduce itself in the environment of use or a material that forms a stable film, and particularly preferably a carbon-based material, copper, stainless steel, aluminum, nickel, titanium, and an alloy thereof.

In addition, a material obtained by coating a surface of a powder material such as silica or acrylic beads with these conductive materials can also be used.

An average particle size of the conductive auxiliary agent is not particularly limited, and is preferably 0.01 μm or more, more preferably 0.02 μm or more, and still more preferably 0.03 μm or more. In addition, the average particle size of the conductive auxiliary agent is preferably 50 μm or less, more preferably 10 μm or less, and still more preferably 5 μm or less.

A shape of the conductive auxiliary agent is not particularly limited, and may be a sphere, a polyhedron, a cylinder, a cone, a cylinder, a pyramid, a prism, or the like.

As the conductive auxiliary agent, conductive fibers can also be used. As the conductive fiber, for example, carbon fibers such as PAN-based carbon fibers or pitch-based carbon fibers, conductive fibers in which a conductive metal or a carbon-based material is dispersed in fibers, conductive fibers in which a fiber surface is coated with a conductive material, and the like can be used.

(Binder)

The binder is not particularly limited as long as it can bind and fix the active material, the conductive auxiliary agent, and the current collector of the electrode. For example, starch, polyvinylidene fluoride, carboxymethyl cellulose, polyvinylpyrrolidone, tetrafluoroethylene, styrene-butadiene rubber, polyethylene, polypropylene, polyethylene glycol, polypropylene glycol, an acrylic resin (polymethyl methacrylate, polyacrylic acid, or the like), a vinyl resin (polyvinyl acetate, polyvinyl alcohol, or the like), a urethane resin, a polyester resin, a polyamide resin, an epoxy resin, a polyimide resin, a silicone resin, a phenol resin, a melamine resin, a urea resin, an aniline resin, an ionomer resin, polycarbonate, and the like can be used.

(First Electrode)

In the first electrode 10₁, electrons are consumed and a reduction reaction occurs. Therefore, the standard electrode potential of the first electrode 10₁ itself is preferably large. In addition, the standard electrode potential of the first electrode 10₁ is preferably larger than the standard electrode potential of the second electrode 10₂. The standard electrode potential of the first electrode 10₁ is preferably −300 mV or more, more preferably 0 V or more, and still more preferably +200 mV or more. In addition, the standard electrode potential of the first electrode 10₁ is preferably 3.5 V or less, more preferably 2.5 V or less, and still more preferably 1.5 V or less.

An electrical resistance of the first electrode 10₁ is preferably 100 kΩcm or less, more preferably 1 kΩcm or less, and still more preferably 10 Ωcm or less, in order to convert a minute signal into a voltage with high sensitivity.

(Active Material of First Electrode)

In the first electrode 10₁, a reduction of the active material of the first electrode 10₁ occurs.

A standard electrode potential of the active material of the first electrode 10₁ is preferably −300 mV or more, more preferably 0 mV or more, and still more preferably +200 mV or more. In addition, the standard electrode potential of the active material of the first electrode 10₁ is preferably 3.5 V or less, more preferably 2.5 V or less, and still more preferably 1.5 V or less.

The active material of the first electrode 10₁ is not particularly limited as long as it is a material having the standard electrode battery, and an organic material, an inorganic material, or an organic-inorganic composite can be used. As a specific material, a substance having an appropriate standard electrode potential can be used and selected from materials described in published documents such as papers, patents, and electrochemical handbooks.

In particular, manganese oxide (MnO₂, Mn₂O₃, MnO(OH), MnO, Mn₃O₄, MnO₃, Mn₂O₇, or the like), silver oxide (AgO₂ or the like), oxygen, ozone, lead oxide (PbO₂ or the like), nickel oxide (Ni₂O₃ or the like), nickel hydroxide (Ni(OH)₂ or the like), nickel oxyhydroxide (NiO(OH) or the like), copper oxide (Cu₂O, CuO, or the like), chromium oxide (CrO, Cr₂O₃, CrO₂, CrO₃, or the like), and iron oxide (Fe₂O₃, FeO, Fe₃O₄, or the like) are preferable because these materials are stably present in the air.

In addition, an active material of the first electrode 10₁ used for a lithium ion battery, a sodium ion battery, a calcium ion battery, a magnesium battery, or the like can be used. Specifically, a metal oxide composed of an alkali metal or an alkaline earth metal and other metals (Co, Ni, Mn, Fe, Mg, Al, and the like) is exemplified.

As the first electrode 10₁, it is preferable to use a material containing gold, silver, copper, platinum, or carbon in terms of high chemical stability in the air.

(Second Electrode)

In the second electrode 10₂, the active material of the second electrode 10₂ is oxidized to release electrons. Therefore, it is preferable to use a substance having a small standard electrode potential in the second electrode 10₂. In addition, the standard electrode potential of the second electrode 10₂ is preferably smaller than the standard electrode potential of the first electrode 10₁. The standard electrode potential of the second electrode 10₂ is preferably −200 mV or less, more preferably −500 mV or less, and still more preferably −700 mV or less. In addition, the standard electrode potential of the second electrode 10₂ is preferably −3.5 V or more, more preferably −2.5 V or more, and still more preferably −1.5 V or more.

An electrical resistance of the second electrode 10₂ is preferably 100 kΩcm or less, more preferably 10 kΩcm or less, and still more preferably 1 kΩcm or less, in order to convert a minute signal into a voltage with high sensitivity.

(Active Material of Second Electrode)

In the second electrode 10₂, a reduction of the active material of the second electrode 10₂ occurs.

A standard electrode potential of the active material of the second electrode 10₂ is preferably −200 mV or less, more preferably −500 mV or less, and still more preferably −700 mVmV or less. In addition, the standard electrode potential of the active material of the second electrode 10₂ is preferably −3.5 V or more, more preferably −2.5 V or more, and still more preferably −1.5 V or more. When the standard electrode potential of the active material of the second electrode 10₂ is −1.5 V or more, water can be used substantially without being electrolyzed.

The active material of the second electrode 10₂ is not particularly limited as long as it is a material having the standard electrode battery, and an organic material, an inorganic material, or an organic-inorganic composite can be used. As a specific material, a substance having an appropriate standard electrode potential can be used and selected from materials described in published documents such as papers, patents, and electrochemical handbooks.

In particular, Zn, Pb, Cd, Mg, a hydrogen storage alloy, methanol, hydrazine, hydrogen, carbon monoxide, formic acid, an amino carboxylic acid-based chelating agent (ethylenediaminetetraacetic acid or the like), and the like are preferable because these materials have high chemical stability in the air.

In addition, an active material used for a lithium ion battery, a sodium ion battery, a calcium ion battery, a magnesium battery, or the like can be used. Specifically, a carbon-based material (hard carbon, non-graphitizable carbon, amorphous carbon, a resin sintered body, cokes, silicon carbide, or the like), a conductive polymer (polythiophene, polyaniline, polypyrrole, polyacetylene, polyphenylene vinylene, PEDOT, or the like), a metal (Li, Sn, Si, Al, Zr, Mg, Ti, or the like), and an alloy thereof, a metal oxide (titanium oxide, lithium-titanium oxide, silicon oxide, or the like), and a material obtained by combining the metal oxide with an alkali metal or an alkaline earth metal are preferable because these materials have high reactivity.

As the second electrode 10₂, a material containing zinc, aluminum, magnesium, chromium, titanium, tin, iron, lithium, or sodium (for example, carbon supporting lithium or sodium or the like) and the like can be preferably used.

(Other Electrode Materials)

As the active material of the electrode 10, a pigment can be used. As the pigment, a natural pigment and a synthetic pigment can be used, and a natural pigment having a small environmental load is more preferable. Examples of the pigment include an azoic dye, an azo dye, acridine, aniline black, indanthrene, eosin, congo red, dihydrointole, methylene blue, a phenazine derivative pigment, neutral red, phenolphthalein, fuchsine, fluorescein, para red, mauve, carotenoid (carotene, xanthophyll, cryptoxanthin, zeaxanthin, fucoxanthin, lycopene, lutein, or the like), flavonoid (flavones, flavanone, anthochlor, anthocyanin, catechin, or the like), quinones (melanin and the like), a porphyrin pigment (chlorophyll, chlorophyllide, bacteriochlorophyll, cytochrome, pheophorbide, pheoporphyrin, hemerythrin, hemoglobin, hemovanadin, hemocyanin, porphyrin, purphin, myoglobin, or the like), a phycobilin pigment (phycocyanin, phycobilin, phycoerythrin, phytochrome, biliverdin, bilirubin, or the like), alizarin, anthocyan, anthraquinone, indigo, urobilin, erythrocruorin, carthamin, xanthommatin, curcumin, crocetin, chlorin, chlorocruorin, genistein, cochineal, gossypol, commelinin, shikonin, stercobilin, tannin, turacin, bixin, hypericin, pinnaglobin, brazilin, purpurin, betacyanin, berberine, horbilin, mangostin morindin, laminaran, leghemoglobin, litmus, rhodopsin, rhodoxanthin, and rhodomatin.

(Material of Device Including n Electrodes)

In the device including the n electrodes 10, any material described above can be used for the third electrode 10₃ to the n-th electrode 10ₙ.

(Space Between Electrodes)

An electrical resistance between the first electrode 10₁ and the second electrode 10₂ is preferably 10 kΩ or more, more preferably 100 kΩ or more, and still more preferably 1 MΩ or more. When the electrical resistance between the first electrode 10₁ and the second electrode 10₂ is 10 kΩ or more, an electromotive force can be generated when a substance is adsorbed, when the electrical resistance is 100 kΩ or more, a substance having low conductivity can be detected, and when the electrical resistance is 1 MΩ or more, a trace substance can be detected.

In the device including the n electrodes 10ₙ, as for an electrical resistance between any two electrodes 10, the description regarding the electrical resistance between the first electrode 10₁ and the second electrode 10₂ can be adopted within a required range.

The resistance between the electrodes can be measured using, for example, a potentiostat or a galvanostat by an alternating current resistance method or the like.

A space between any two electrodes may be a vacuum, or may be filled with a substance such as a gas, a liquid, or a solid. In order to keep the distance between the electrodes constant and to stabilize the measured value, it is preferable that the electrodes 10 are formed on the surface of the substrate 100.

(Substrate)

The first electrode 10₁ and the second electrode 10₂ may be formed on the surface of the substrate 100, may be formed inside the substrate 100, and may exist independently in the air. The first electrode 10₁ and the second electrode 10₂ are formed on the surface of or inside the substrate 100, such that the distance between the first electrode 10₁ and the second electrode 10₂ becomes constant. In a case where the first electrode 10₁ and the second electrode 10₂ are formed on the surface of or inside the substrate 100, the first electrode 10₁ and the second electrode 10₂ are physically connected via the substrate 100.

Even in the device including the n electrodes, the n electrodes from the first electrode 10₁ to the n electrode 10ₙ may be formed on the surface of the substrate 100, may be formed inside the substrate 100, or may exist independently in the air. The electrodes 10 are formed on the surface of or inside the substrate 100, such that distances between the electrodes 10 become constant.

The substrate 100 is not particularly limited as long as it can support the electrodes 10, and an organic material, an inorganic material, or an organic-inorganic composite material can be used.

As the organic material, specifically, polyethylene terephthalate (PET), polyethylene naphthalate (PEN), polycarbonate (PC), a cycloolefin polymer (COP), polyimide (PI), silicone, paper phenol, paper epoxy, and Teflon (registered trademark), and the like can be used. In particular, PET and PEN are available at low cost, and are useful and preferable from the viewpoint of business. In addition, polyimide is preferable because it has high heat resistance and chemical resistance and thus can be used in processes such as photolithography and soldering.

As the inorganic material, alumina, ceramic, a composite, glass, thin film glass, a metal foil having a surface on which an oxide film is formed, a silicon wafer having a surface on which an oxide film is formed, and the like can be used. In particular, glass and a silicon wafer can be preferably used because it is easy to process a metal on a surface thereof.

As the organic-inorganic composite material, for example, glass epoxy, a glass composite, an organic material in which an inorganic filler is dispersed, an organic material having a surface coated with an inorganic layer, and the like can be used. Examples of a coating method of the inorganic layer include a sol-gel method, a vapor deposition method, a sputtering method, a chemical vapor deposition (CVD) method, and an atomic layer deposition (ALD) method.

As the substrate 100, a hydrophilic material can also be used. Since the substrate 100 using a hydrophilic material easily adsorbs water or a water-soluble component in the air, it is possible to detect water or a water-soluble component with high sensitivity.

A surface free energy of the substrate 100 using a hydrophilic material is preferably 32 mJ/m² or more, more preferably 40 mJ/m² or more, and still more preferably 45 mJ/m² or more. When the surface free energy of the substrate 100 is within the above range, water or a water-soluble component can be detected. In addition, the surface free energy of the substrate 100 using a hydrophilic material is preferably 2,000 mJ/m² or less, more preferably 1,400 mJ/m² or less, and still more preferably 70 mJ/m² or less. When the surface free energy of the substrate 100 is within the above range, it is possible to desorb the adsorbed water or water-soluble component.

When a hydrophilic functional group (a hydroxyl group, an amino group, an imino group, a thiol group, a sulfonic acid group, a phosphonic acid group, a phosphonic acid ester group, a functional group having a succinimide skeleton, a functional group having a pyrrolidone skeleton, a selenol group, a polysulfide group, a polyselenide group, a carboxy group, a functional group having an acid anhydride skeleton, a nitro group, a cyano group, or the like) is present on the surface of the substrate 100, water or a water-soluble component can be detected with high sensitivity.

A concentration of the hydrophilic functional group on the surface of the substrate 100 is preferably 0.1 atomic % or more, more preferably 1.0 atomic % or more, and still more preferably 10 atomic % or more. In addition, the concentration of the hydrophilic functional group on the surface of the substrate 100 is preferably 90 atomic % or less, more preferably 50 atomic % or less, and still more preferably 40 atomic % or less. When the concentration of the hydrophilic functional group on the surface of the substrate 100 is within the above range, a short circuit between the first electrode 10₁ and the second electrode 10₂ can be prevented. The concentration of the hydrophilic functional group on the surface of the substrate 100 can be quantified by ESCA analysis by a gas phase chemical modification method.

As the substrate 100, a water-repellent material can also be used. In addition, as the substrate 100, a substrate 100 having a surface on which a water-repellent material is formed can be used. The water-repellent material may be formed on the entire surface of the substrate or may be formed only in the vicinity of the electrode. Since the substrate 100 using a water-repellent material easily adsorbs an organic solvent or a fat-soluble component in the air, it is possible to detect an organic solvent or a fat-soluble component with high sensitivity.

A surface free energy of the substrate 100 using a water-repellent material is preferably 10 mJ/m² or more, more preferably 20 mJ/m² or more, and still more preferably 25 mJ/m² or more. When the surface free energy of the substrate 100 is within the above range, it is possible to desorb the adsorbed fat-soluble component. In addition, the surface free energy of the substrate 100 using a water-repellent material is preferably 2,000 mJ/m² or less, more preferably 1,400 mJ/m² or less, and still more preferably 70 mJ/m² or less. When the surface free energy of the substrate 100 is within the above range, a fat-soluble component can be detected.

A contact angle of water to the substrate 100 is preferably 90 degrees or less, more preferably 85 degrees or less, and still more preferably 75 degrees or less.

Since an effective distance between the first electrode 10₁ and the second electrode 10₂ is smaller as an arithmetic average roughness Ra of the surface of the substrate 100 is smaller, the sensitivity is improved. The arithmetic average roughness Ra of the surface of the substrate 100 is preferably 1.0 μm or less, and more preferably 0.10 μm or less, and still more preferably 0.010 μm or less. In addition, as the arithmetic average roughness Ra of the surface of the substrate 100 is increased, adhesion to other layers is improved. The arithmetic average roughness Ra of the surface of the substrate 100 is preferably 1 nm or more.

A thickness of the substrate 100 is not particularly limited. The thickness of the substrate 100 is preferably 1 μm or more and more preferably 10 μm or more. In addition, the thickness of the substrate 100 is preferably 5 mm or less, more preferably 2 mm or less, and still more preferably 1 mm or less. When the thickness is 1 μm or more, the film can be used as a free-standing film, and when the thickness is 10 μm or more, a high-strength film is obtained and handling is facilitated. When the thickness is more than 5 mm, the weight of the device increases and installability is deteriorated, and thus the thickness is preferably 5 mm or less. The thickness is preferably 1 mm or less because a lightweight element can be formed, and the thickness is preferably 200 μm or less because flexibility is enhanced and it is difficult to be broken by bending.

An electrical conductivity or ionic conductivity of the substrate 100 is not particularly limited. The electrical conductivity or ionic conductivity of the substrate 100 is preferably 1 S/cm or less, more preferably 10⁻³ S/cm or less, and still more preferably 10⁻⁵ S/cm or less. When the electrical conductivity or ionic conductivity of the substrate 100 is 1 S/cm or less, an electromotive force can be generated when a substance is adsorbed, when the electrical conductivity or ionic conductivity is 10⁻³ S/cm or less, a substance having low conductivity can be detected, and when the electrical conductivity or ionic conductivity is 10⁻⁵ S/cm or less, a trace substance can be detected.

(Sensor)

The device described above can be used as a sensor. In the device described above, a potential difference occurs between the first electrode 10₁ and the second electrode 10₂ due to adsorption of a substance. Adsorption and desorption of a substance can be detected by measuring the potential difference with a voltmeter.

Since the amount of electric charge generated by adsorption of a substance is significantly small, the voltmeter preferably has a high input impedance. Specifically, the input impedance is preferably 10 MΩ or more, more preferably 100 MΩ or more, and still more preferably 10 GΩ or more.

(Sensor Including n Electrodes)

The device including three electrodes 10 and the device including n electrodes 10 described above can be used as sensors.

Adsorption and desorption of a substance can be sensed by measuring a potential difference between the k-th electrode 10ₖ and the j-th electrode 10ⱼ with a voltmeter.

{n(n−1)/2}-dimensional data can be obtained by measuring a voltage between any two electrodes using a sensor including n electrodes such that more pieces of information on the adsorbed and desorbed substance can be obtained.

Among the n electrodes 10 from the first electrode 10₁ to the n-th electrode 10ₙ, three or more electrodes 10 may be formed of different materials. Different pieces of information can be obtained from electrodes 10 formed of different materials. Therefore, the sensor including the n electrodes 10 preferably includes three or more electrodes 10 formed of different materials. For example, in a case of a sensor including a first electrode 10₁, a second electrode 10₂, and a third electrode 10₃, in which a material of the first electrode 10₁, a material of the second electrode 10₂, and a material of the third electrode 10₃ are different from each other, since a response to the adsorbed substance differs between a set of the first electrode 10₁ and the second electrode 10₂ and a set of the first electrode 10₁ and the third electrode 10₃, a plurality of pieces of information on the characteristics of the adsorbed substance can be obtained. For example, by measuring a current, a voltage, and the like generated when a substance is adsorbed between the first electrode 10₁ and the second electrode 10₂ and the adsorbed substance comes into contact with both of these electrodes, and a current, a voltage, and the like generated when a substance is adsorbed between the first electrode 10₁ and the third electrode 10₃ and the adsorbed substance comes into contact with both of these electrodes, it is possible to more accurately identify the adsorbed substance.

The shortest distance between the k-th electrode 10ₖ and the j-th electrode 10ⱼ may be constant or different for any k and j. Different pieces of information can be obtained from sets of electrodes 10 having the shortest distances between the electrodes that are different from each other. Therefore, the sensor including the n electrodes 10 preferably includes a plurality of sets of electrodes 10 having the shortest distances between the electrodes that are different from each other. For example, in a case of a sensor including a first electrode 10₁, a second electrode 10₂, and a third electrode 10₃, in which values of the shortest distance between the first electrode 10₁ and the second electrode 10₂ and the shortest distance between the first electrode 10₁ and the third electrode 10₃ are different from each other, since a size of a substance that can be detected differs between a set of the first electrode 10₁ and the second electrode 10₂ and a set of the first electrode 10₁ and the third electrode 10₃, information on the size of the adsorbed substance can be obtained.

Among the n electrodes 10 from the first electrode 10₁ to the n-th electrode 10ₙ, two or more and (n−1) or fewer electrodes 10 may have the same shape, material, and physical properties.

(System Including Plurality of Sensors)

A system including a plurality of sensors will be described. A sensor system includes at least two sensors, a first sensor includes a first electrode 10₁ and a second electrode 10₂, the first electrode 10₁ and the second electrode 10₂ are not electrically connected, the shortest distance between the first electrode 10₁ and the second electrode 10₂ is 0.001 μm or more and 100 μm or less, an absolute value of a difference between a standard electrode potential of the first electrode 10₁ and a standard electrode potential of the second electrode 10₂ is 0.1 V or more, a second sensor includes a third electrode 10₃ and a fourth electrode 10₃, the third electrode 10₃ and the fourth electrode 10₄ are not electrically connected, the shortest distance between the third electrode 10₃ and the fourth electrode 10₄ is 0.001 μm or more and 100 μm or less, and an absolute value of a difference between a standard electrode potential of the third electrode 10₃ and a standard electrode potential of the fourth electrode 10₄ is 0.1 V or more.

In the sensor system, the shortest distance between the first electrode 10₁ and the second electrode 10₂ may be different from the shortest distance between the third electrode 10₃ and the fourth electrode 10₄.

In the sensor system, a combination of a material constituting the first electrode 10₁ and a material constituting the second electrode 10₂ may be different from a combination of a material constituting the third electrode 10₃ and a material constituting the fourth electrode 10₄.

(Sensing Method)

When a particle containing an ionized molecule comes into contact with the first electrode 10₁ and the second electrode 10₂, an oxidation-reduction reaction occurs on the first electrode 10₁ and the second electrode 10₂, and a minute potential difference occurs. By measuring the potential difference, it is possible to detect whether a substance is adsorbed to the first electrode 10₁ and the second electrode 10₂.

In the case of the device including the n electrodes 10, sensing can be performed by bringing the particle containing ionized molecule into contact with any two electrodes 10.

The particle containing an ionized molecule may be solid, liquid, aerosol, or gas. A conventional sensor element detects a substance by bringing large solid or liquid droplet into contact between electrodes, but the sensor of the present embodiment can also detect extremely minute solid particle, liquid droplet, aerosol, and gas of 100 μm or less.

The particle containing an ionized molecule cannot contain a molecule larger than themselves, such that a molecule smaller than the particle containing an ionized molecule can be selectively sensed.

A particle size of the particle containing an ionized molecule is preferably 100 μm or less, more preferably 50 μm or less, still more preferably 20 μm or less, particularly preferably 10 μm or less, particularly preferably 1 μm or less, particularly preferably 0.8 μm or less, and most preferably 0.5 μm or less. When the particle size of the particle containing an ionized molecule is 100 μm or less, an allergen such as pollen can be detected, when the particle size is 10 μm or less, a harmful aerosol such as PM 10 can be selectively detected, and when the particle size is 1 μm or less, a nanoparticle harmful to the human body can be selectively detected.

(Power Generation Method)

The device of the present embodiment generates an electromotive force by contact of the particle containing an ionized molecule. The electromotive force can be used as energy. Specifically, in the device including the first electrode 10₁ and the second electrode 10₂ in which the first electrode 10₁ and the second electrode 10₂ are not electrically connected, the shortest distance between the first electrode 10₁ and the second electrode 10₂ is 0.001 μm or more and 100 μm or less, and the absolute value of the difference between the standard electrode potential of the first electrode 10₁ and the standard electrode potential of the second electrode 10₂ is 0.1 V or more, electric power can be generated by bringing the particle having a size of 100 μm or less and containing an ionized molecule into contact with the first electrode 10₁ and the second electrode 10₂.

For the particle size of the particle containing an ionized molecule related to the power generation method, the description related to the sensing method described above can be adopted within a required range.

(Power Generation Method Using Plurality of Devices)

Larger energy can be generated by using a plurality of devices described above. By short-circuiting a second electrode of a b-th device and a first electrode of a (b+1)-th device using a devices, it is possible to increase the electromotive force when the particle containing an ionized molecule comes into contact with the electrodes. Here, a is an integer of 2 or more, and b is an integer of 1 or more and (a−1) or less.

(Formation Method of Electrode)

A formation method of the electrode is not particularly limited, and for example, various methods such as vapor deposition, electrolytic plating, electroless plating, coating, laser ablation, cutting, printing, photolithography, imprinting, and bonding can be used.

### EXAMPLES

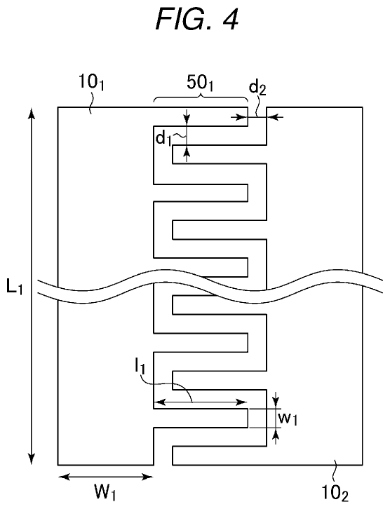

FIG. 4 is a view for explaining an arrangement of electrodes in an Example according to an embodiment of the present invention. FIG. 4 illustrates an arrangement of electrodes of a device including two electrodes of a first electrode 10₁ and a second electrode 10₂.

As illustrated in FIG. 4, the first electrode 10₁ and the second electrode 10₂ have a comb shape. The shapes of the first electrode 10₁ and the second electrode 10₂ are in a congruent relationship.

The first electrode 10₁ and the second electrode 10₂ are arranged so that comb teeth are engaged with each other. As illustrated in the drawing, a distance between the electrodes of convex portions of the comb teeth of the first electrode 10₁ and the second electrode 10₂ is defined as an inter-electrode distance d₁. A distance between the electrodes of convex portions and concave portions of the comb teeth of the first electrode 10₁ and the second electrode 10₂ is defined as an inter-electrode distance d₂.

At a portion where the comb teeth of the first electrode 10₁ and the second electrode 10₂ are engaged with each other, the inter-electrode distance d₁ is constant. In addition, at the portion where the comb teeth of the first electrode 10₁ and the second electrode 10₂ are engaged with each other, the inter-electrode distance d₂ is constant.

As illustrated in the drawing, a length of the first electrode 10₁ in a direction perpendicular to an orientation of the comb teeth is defined as a length L₁ of the first electrode 10₁. A length of a portion of the first electrode 10₁ that is not a comb tooth in a direction parallel to the orientation of the comb teeth is defined as a width W₁ of the first electrode 10₁. Hereinafter, a length of the electrode 10 in the direction perpendicular to the orientation of the comb teeth is defined as a length L of the electrode 10. In addition, a length of a portion of the electrode that is not a comb tooth in the direction parallel to the orientation of the comb teeth is defined as a width W of the electrode 10.

In addition, as described in the drawing, a length of a convex portion 50₁ of the comb teeth of the first electrode 10₁ in the direction parallel to the orientation of the comb teeth is defined as a length l₁ of the convex portion 50₁ of the comb teeth of the first electrode 10₁. A length of the convex portion 50₁ of the comb teeth of the first electrode 10₁ in the direction perpendicular to the orientation of the comb teeth is defined as a width w₁ of the convex portion 50₁ of the comb teeth of the first electrode 10₁. Hereinafter, a length of the convex portion 50 of the comb teeth of the electrode 10 in the direction parallel to the orientation of the comb teeth is defined as a length l of the convex portion 50 of the comb teeth of the electrode 10. A length of the convex portion 50 of the comb teeth of the electrode 10 in the direction perpendicular to the orientation of the comb teeth is defined as a width w of the convex portion 50 of the comb teeth of the electrode 10.

In Examples 1 to 6 and 8 and Comparative Examples 1 and 2, the device having the arrangement of the electrodes as illustrated in FIG. 4 was manufactured. Hereinafter, a device having a structure in which comb-shaped electrodes are engaged with each other is referred to as a device having a comb-shaped electrode structure.

(Evaluation Method)

A potential difference between the electrodes was measured with an oscilloscope having an input impedance of 10 MΩ when the manufactured device was exposed to an atomizer, steam of a humidifier, exhalation, and air having a humidity of 90%.

### Example 1

A first electrode 10₁ and a second electrode 10₂ were formed on a surface of a substrate 100 formed of glass to manufacture a device having a comb-shaped electrode structure. A thickness of the substrate 100 was 700 μm. An adhesive layer 20₁ formed of titanium was formed between the substrate 100 and a conductive layer 30₁ of the first electrode 10₁. In addition, an adhesive layer 20₂ formed of titanium was formed between the substrate 100 and a conductive layer 30₂ of the second electrode 10₂. A thickness of each of the adhesive layer 20₁ and the adhesive layer 20₂ was 10 nm. Gold was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 4 mm, a width W of the electrode 10 was 1.5 mm, a thickness of the electrode 10 was 0.1 μm, a length l of a convex portion of comb teeth of the electrode 10 was 4 mm, and a width w of the convex portion 50 of the comb teeth of the electrode 10 was 10 μm. In addition, an inter-electrode distance d₁ was 5 μm, and an inter-electrode distance d₂ was 500 μm. The number of the convex portions 50 of the comb teeth of each of the first electrode 10₁ and the second electrode 10₂ was 66, respectively. The details of the device are shown in Tables 1 and 2.

The voltages of 0.6 V for the atomizer, 0.6 V for the steam of the humidifier, 0.6 V for the exhalation, and 0.6 V for the air having a humidity of 90% were observed. The evaluation results are shown in Table 2.

### Example 2

A first electrode 10₁ and a second electrode 10₂ were formed on a surface of a substrate 100 formed of epoxy glass to manufacture a device having a comb-shaped electrode structure. A thickness of the substrate 100 was 1,600 μm. Copper was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 5 mm, a width W of the electrode 10 was 4 mm, a thickness of the electrode 10 was 17 μm, a length l of a convex portion 50 of comb teeth of the electrode 10 was 2.5 mm, and a width w of the convex portion 50 of the comb teeth of the electrode 10 was 100 μm. In addition, an inter-electrode distance d₁ was 100 μm, and an inter-electrode distance d₂ was 100 μm. The number of the convex portions 50 of the comb teeth of each of the first electrode 10₁ and the second electrode 10₂ was 10, respectively. The details of the device are shown in Tables 1 and 2.

The voltages of 0.7 V for the atomizer, 0 V for the steam of the humidifier, 0 V for the exhalation, and 0 V for the air having a humidity of 90% were observed. The evaluation results are shown in Table 2.

### Example 3

A first electrode 10₁ and a second electrode 10₂ were formed on a surface of a substrate 100 formed of epoxy glass to manufacture a device having a comb-shaped electrode structure. A thickness of the substrate 100 was 1,600 μm. Copper was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 5 mm, a width W of the electrode 10 was 4 mm, a thickness of the electrode 10 was 17 μm, a length l of a convex portion 50 of comb teeth of the electrode 10 was 2.5 mm, and a width w of the convex portion 50 of the comb teeth of the electrode 10 was 100 μm. In addition, an inter-electrode distance d 1 was 75 μm, and an inter-electrode distance d 2 was 100 μm. The number of the convex portions 50 of the comb teeth of each of the first electrode 10₁ and the second electrode 10₂ was 10, respectively. The details of the device are shown in Tables 1 and 2.

The voltages of 0.7 V for the atomizer, 0.7 V for the steam of the humidifier, 0 V for the exhalation, and 0 V for the air having a humidity of 90% were observed. The evaluation results are shown in Table 2.

### Example 4

A first electrode 10₁ and a second electrode 10₂ were formed on a surface of a substrate 100 formed of polyimide to manufacture a device having a comb-shaped electrode structure. A thickness of the substrate 100 was 75 μm. An adhesive layer 20₁ formed of nichrome was formed between the substrate 100 and a conductive layer 30₁ of the first electrode 10₁. In addition, an adhesive layer 20₂ formed of nichrome was formed between the substrate 100 and a conductive layer 30₂ of the second electrode 10₂. A thickness of each of the adhesive layer 20₁ and the adhesive layer 20₂ was 10 nm. Copper was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 4 mm, a width W of the electrode 10 was 4 mm, a thickness of the electrode 10 was 2 μm, a length l of a convex portion 50 of comb teeth of the electrode 10 was 2 mm, and a width w of the convex portion 50 of the comb teeth of the electrode 10 was 100 μm. In addition, an inter-electrode distance d₁ was 10 μm, and an inter-electrode distance d₂ was 100 μm. The number of the convex portions of the comb teeth of each of the first electrode 10₁ and the second electrode 10₂ was 10, respectively. The details of the device are shown in Tables 1 and 2.

The voltages of 0.7 V for the atomizer, 0.7 V for the steam of the humidifier, 0.7 V for the exhalation, and 0.7 V for the air having a humidity of 90% were observed. The evaluation results are shown in Table 2.

### Example 5

A first electrode 10₁ and a second electrode 10₂ were formed on a surface of a substrate 100 formed of polyimide to manufacture a device having a comb-shaped electrode structure. A thickness of the substrate 100 was 75 μm. An adhesive layer 20₁ formed of nichrome was formed between the substrate 100 and a conductive layer 30₁ of the first electrode 10₁. In addition, an adhesive layer 20₂ formed of nichrome was formed between the substrate 100 and a conductive layer 30₂ of the second electrode 10₂. A thickness of each of the adhesive layer 20₁ and the adhesive layer 20₂ was 10 nm. Copper was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 4 mm, a width W of the electrode 10 was 4 mm, a thickness of the electrode 10 was 2 μm, a length l of a convex portion 50 of comb teeth of the electrode 10 was 2 mm, and a width w of the convex portion 50 of the comb teeth of the electrode 10 was 100 μm. In addition, an inter-electrode distance d₁ was 20 μm, and an inter-electrode distance d₂ was 100 μm. The number of the convex portions 50 of the comb teeth of each of the first electrode 10₁ and the second electrode 10₂ was 10, respectively. The details of the device are shown in Tables 1 and 2.

The voltages of 0.7 V for the atomizer, 0.7 V for the steam of the humidifier, 0.7 V for the exhalation, and 0 V for the air having a humidity of 90% were observed. The evaluation results are shown in Table 2.

### Example 6

A first electrode 10₁ and a second electrode 10₂ were formed on a surface of a substrate 100 formed of polyimide to manufacture a device having a comb-shaped electrode structure. A thickness of the substrate 100 was 75 μm. An adhesive layer 20₁ formed of nichrome was formed between the substrate 100 and a conductive layer 30₁ of the first electrode 10₁. In addition, an adhesive layer 20₂ formed of nichrome was formed between the substrate 100 and a conductive layer 30₂ of the second electrode 10₂. A thickness of each of the adhesive layer 20₁ and the adhesive layer 20₂ was 10 nm. Copper was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 4 mm, a width W of the electrode 10 was 4 mm, a thickness of the electrode 10 was 2 μm, a length l of a convex portion 50 of comb teeth of the electrode 10 was 2 mm, and a width w of the convex portion 50 of the comb teeth of the electrode 10 was 100 μm. In addition, an inter-electrode distance d₁ was 50 μm, and an inter-electrode distance d₂ was 100 μm. The number of the convex portions of the comb teeth of each of the first electrode 10₁ and the second electrode 10₂ was 10, respectively. The details of the device are shown in Tables 1 and 2.

### Example 7

The voltages of 0.7 V for the atomizer, 0.7 V for the steam of the humidifier, 0 V for the exhalation, and 0 V for the air having a humidity of 90% were observed. The evaluation results are shown in Table 2.

### Example 7

Copper was used as a material of a conductive layer 30₁ of a first electrode 10₁, and zinc was used as a material of a conductive layer 30₂ of a second electrode 10₂. Both the first electrode 10₁ and the second electrode 10₂ had a plate shape with a length of 10 mm, a width of 10 mm, and a thickness of 100 μm, and were laminated in parallel so that surfaces having large surface areas faced each other and the first electrode and the second electrode were not in contact with each other, thereby manufacturing a device. A distance between the first electrode 10₁ and the second electrode 10₂ was 10 μm. The details of the device are shown in Tables 1 and 2.

The voltages of 0.7 V for the atomizer, 0.7 V for the steam of the humidifier, 0.7 V for the exhalation, and 0 V for the air having a humidity of 90% were observed. The evaluation results are shown in Table 2.

### Comparative Example 1

A first electrode 10₁ and a second electrode 10₂ were formed on a surface of a substrate 100 formed of epoxy glass to manufacture a device having a comb-shaped electrode structure. A thickness of the substrate 100 was 1,600 μm. Copper was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 5 mm, a width W of the electrode 10 was 4 mm, a thickness of the electrode 10 was 17 μm, a length l of a convex portion 50 of comb teeth of the electrode 10 was 2.5 mm, and a width w of the convex portion 50 of the comb teeth of the electrode 10 was 100 μm. In addition, an inter-electrode distance d₁ was 150 μm, and an inter-electrode distance d 2 was 100 μm. The number of the convex portions 50 of the comb teeth of each of the first electrode 10₁ and the second electrode 10₂ was 10, respectively. The details of the device are shown in Tables 1 and 2.

No voltage was observed for any of the atomizer, the steam of the humidifier, the exhalation, and the air having a humidity of 90%. The evaluation results are shown in Table 2.

### Comparative Example 2

A first electrode 10₁ and a second electrode 10₂ were formed on a surface of a substrate 100 formed of polyimide to manufacture a device having a comb-shaped electrode structure. A thickness of the substrate 100 was 75 μm. An adhesive layer 20₁ formed of nichrome was formed between the substrate 100 and a conductive layer 30₁ of the first electrode 10₁. In addition, an adhesive layer 20₂ formed of nichrome was formed between the substrate 100 and a conductive layer 30₂ of the second electrode 10₂. A thickness of each of the adhesive layer 20₁ and the adhesive layer 20₂ was 10 nm. Copper was used as a material of each of the conductive layer 30₁ of the first electrode 10₁ and the conductive layer 30₂ of the second electrode 10₂. An active material layer 40₂ formed of nickel was formed on a surface of the conductive layer 30₂ of the second electrode 10₂ opposite to the adhesive layer 20₂. A thickness of the active material layer 40₂ was 2 μm. A length L of the electrode 10 was 4 mm, a width W of the electrode 10 was 4 mm, a thickness of the electrode 10 was 2 μm, a length l of a convex portion 50 of comb teeth of the electrode 10 was 2 mm, and a width w of the convex portion 50 of the comb teeth of the electrode 10 was 100 μm. In addition, an inter-electrode distance d₁ was 10 μm, and an inter-electrode distance d₂ was 100 μm. The number of the convex portions of the comb teeth of each of the first electrode 10₁ and the second electrode 10₂ was 10, respectively. The details of the device are shown in Tables 1 and 2.

No voltage was observed for any of the atomizer, the steam of the humidifier, the exhalation, and the air having a humidity of 90%. The evaluation results are shown in Table 2.

TABLE 1

| | | | First electrode | | | | | | Second electrode | | | | | |
|---|---|---|---|---|---|---|---|---|---|---|---|---|---|---|
| | Adhesive layer of | | Conductive | | Active material | | Adhesive layer of | | Conductive | | Active material | | | |
| | first electrode | | layer | | layer | | second electrode | | layer | | layer | | Substrate | |
| | | Thick- | | Thick- | | Thick- | | Thick- | | Thick- | | Thick- | | Thick- |
| | Mate- | ness | Mate- | ness | Mate- | ness | Mate- | ness | Mate- | ness | Mate- | ness | Mate- | ness |
| | rial | (nm) | rial | (μm) | rial | (μm) | rial | (μm) | rial | (μm) | rial | (μm) | rial | (μm) |
| Example 1 | Tita- | 10 | Gold | 0.1 | — | — | Tita- | 10 | Gold | 0.1 | Zinc | 0.5 | Glass | 700 |
| | nium | | | | | | nium | | | | | | | |
| Example 2 | — | — | Copper | 17 | — | — | — | — | Copper | 17 | Zinc | 0.5 | Epoxy | 1600 |
| | | | | | | | | | | | | | glass | |
| Example 3 | — | — | Copper | 17 | — | — | — | — | Copper | 17 | Zinc | 0.5 | Epoxy | 1600 |
| | | | | | | | | | | | | | glass | |
| Example 4 | Ni- | 10 | Copper | 2 | — | — | Ni- | 10 | Copper | 2 | Zinc | 0.5 | Polyimide | 75 |
| | chrome | | | | | | chrome | | | | | | | |
| Example 5 | Ni- | 10 | Copper | 2 | — | — | Ni- | 10 | Copper | 2 | Zinc | 0.5 | Polyimide | 75 |
| | chrome | | | | | | chrome | | | | | | | |
| Example 6 | Ni- | 10 | Copper | 2 | — | — | Ni- | 10 | Copper | 2 | Zinc | 0.5 | Polyimide | 75 |
| | chrome | | | | | | chrome | | | | | | | |
| Example 7 | — | — | Copper | 100 | — | — | — | — | Zinc | 100 | — | — | — | — |
| Comparative | — | — | Copper | 17 | — | — | — | — | Copper | 17 | Zinc | 0.5 | Epoxy | 1600 |
| Example 1 | | | | | | | | | | | | | glass | |
| Comparative | Ni- | 10 | Copper | 2 | — | — | Ni- | 10 | Copper | 2 | Nickel | 2 | Polyimide | 75 |
| Example 2 | chrome | | | | | | chrome | | | | | | | |

TABLE 2

| | | Comb-shaped electrode | | | | | | | | | | | |
|---|---|---|---|---|---|---|---|---|---|---|---|---|---|
| | | Number | | | | | | | Facing flat- | Measurement results (V) | | | |
| | | of convex | | | | | | | plate electrode | | | | Air |
| | | portions | Size | | | | | | Inter- | | | | having |
| | Shape of | of comb teeth | L | W | l | w | d₁ | d₂ | electrode | | Steam of | | humidity |
| | electrode | (number) | (mm) | (mm) | (mm) | (μm) | (μm) | (μm) | distance (μm) | Atomizer | humidifier | Exhalation | of 90% |
| Example 1 | Comb | 66 | 4 | 1.5 | 4 | 10 | 5 | 500 | — | 0.6 | 0.6 | 0.6 | 0.6 |
| Example 2 | Comb | 10 | 5 | 4 | 2.5 | 100 | 100 | 100 | — | 0.7 | 0 | 0 | 0 |
| Example 3 | Comb | 10 | 5 | 4 | 2.5 | 100 | 75 | 100 | — | 0.7 | 0.7 | 0 | 0 |
| Example 4 | Comb | 10 | 4 | 4 | 2 | 100 | 10 | 100 | — | 0.7 | 0.7 | 0.7 | 0.7 |
| Example 5 | Comb | 10 | 4 | 4 | 2 | 100 | 20 | 100 | — | 0.7 | 0.7 | 0.7 | 0 |
| Example 6 | Comb | 10 | 4 | 4 | 2 | 100 | 50 | 100 | — | 0.7 | 0.7 | 0 | 0 |
| Example 7 | Facing | — | — | — | — | — | — | — | 10 | 0.7 | 0.7 | 0.7 | 0 |
| | flat plate | | | | | | | | | | | | |
| Comparative | Comb | 10 | 5 | 4 | 2.5 | 100 | 150 | 100 | — | 0 | 0 | 0 | 0 |
| Example 1 | | | | | | | | | | | | | |
| Comparative | Comb | 10 | 4 | 4 | 2 | 100 | 10 | 100 | — | 0 | 0 | 0 | 0 |
| Example 2 | | | | | | | | | | | | | |

### Example 8

A first electrode 10₁ and a second electrode 10₂ were formed on a surface of a substrate 100 formed of glass to manufacture a device having a comb-shaped electrode structure. A thickness of the substrate 100 was 700 μm. An adhesive layer 20₁ formed of titanium was formed between the substrate 100 and a conductive layer 30₁ of the first electrode 10₁. In addition, an adhesive layer 20₂ formed of titanium was formed between the substrate 100 and a conductive layer 30₂ of the second electrode 10₂. A thickness of each of the adhesive layer 20₁ and the adhesive layer 20₂ was 10 nm. Gold was used as a material of each of the conductive layer 30₁ of the first electrode 10₁ and the conductive layer 30₂ of the second electrode 10₂. An active material layer 40₂ formed of zinc was formed on a surface of the conductive layer 30₂ of the second electrode 10₂ opposite to the adhesive layer 20₂. A thickness of the active material layer 40₂ was 0.5 μm. A length L of the electrode 10 was 4 mm, a width W of the electrode 10 was 1.5 mm, a thickness of the electrode 10 was 0.1 μm, a length l of a convex portion 50 of comb teeth of the electrode 10 was 4 mm, and a width w of the convex portion 50 of the comb teeth of the electrode 10 was 10 μm. In addition, an inter-electrode distance d₁ was 5 μm, and an inter-electrode distance d₂ was 500 μm. The number of the convex portions 50 of the comb teeth of each of the first electrode 10₁ and the second electrode 10₂ was 66, respectively.

Four devices were manufactured as a first device, a second device, a third device, and a fourth device. Then, a first electrode of the first device and a second electrode of the second device were electrically connected, a first electrode of the second device and a second electrode of the third device were electrically connected, and a first electrode of the third device and a second electrode of the fourth device were electrically connected. In this state, a potential difference between the second electrode of the first device and the first electrode of the fourth device was measured.

The voltages of 2.4 V for the atomizer, 2.4 V for the steam of the humidifier, 2.4 V for the exhalation, and 2.4 V for the air having a humidity of 90% were observed. Further, when an LED was connected between the second electrode of the first device and the first electrode of the fourth device, the LED was turned on by spraying mist of an atomizer between the electrodes.

### INDUSTRIAL APPLICABILITY

The device can be used as a sensor or a power generation element.

### REFERENCE SIGNS LIST

10 Electrode
20 Adhesive layer
30 Conductive layer
40 Active material layer
50 Convex portion of comb teeth of electrode
100 Substrate

1. A device comprising a first electrode and a second electrode,
wherein the first electrode and the second electrode are not electrically connected,
a shortest distance between the first electrode and the second electrode is 0.001 μm or more and 100 μm or less,
an absolute value of a difference between a standard electrode potential of the first electrode and a standard electrode potential of the second electrode is 0.1 V or more, and
surfaces of the first electrode and the second electrode are partly or entirely exposed.

2. The device according to claim 1, wherein the device includes a substrate, and
the first electrode and the second electrode are physically connected via the substrate.

3. The device according to claim 1, wherein the shortest distance between the first electrode and the second electrode is 10 μm or less.

4. The device according to claim 1, wherein the first electrode and the second electrode have a comb shape.

5. The device according to claim 2, wherein the first electrode and the second electrode are formed on a surface of the substrate.

6. The device according to claim 1, wherein the first electrode contains gold, silver, copper, platinum, or carbon.

7. The device according to claim 1, wherein the second electrode contains zinc, aluminum, magnesium, chromium, titanium, tin, iron, lithium, or sodium.

8. The device according to claim 1, wherein the first electrode and/or the second electrode has a laminated structure of a plurality of materials.

9. The device according to claim 2, wherein the first electrode and/or the second electrode is connected to the substrate via an adhesive layer.

10. The device according to claim 1, wherein an electrical resistance between the first electrode and the second electrode is 10 kΩ or more.

11. The device according to claim 2, wherein an electrical conductivity or ionic conductivity of the substrate is 1 S/cm or less.

12. The device according to claim 2, wherein a surface free energy of the substrate is 32 mJ/m² or more and 2,000 mJ/m² or less.

13. The device according to claim 2, wherein an amount of hydroxyl groups on a surface of the substrate is 0.1 atomic % or more and 90 atomic % or less.

14. The device according to claim 2, wherein a surface free energy of the substrate is 10 mJ/m² or more and 2,000 mJ/m² or less.

15. The device according to claim 2, wherein a water-repellent material is formed on a surface of the substrate.

16. The device according to claim 2, wherein a contact angle of water to a surface of the substrate is 90° or less.

17. The device according to claim 2, wherein an arithmetic average roughness Ra of a surface of the substrate is 0.001 μm or more and 1 μm or less.

18. The device according to claim 2, wherein the substrate is glass, polyimide, PET, PEN, or a silicon wafer.

19. A sensor comprising a first electrode and a second electrode,
wherein the first electrode and the second electrode are not electrically connected,
a shortest distance between the first electrode and the second electrode is 0.001 μm or more and 100 μm or less, and
an absolute value of a difference between a standard electrode potential of the first electrode and a standard electrode potential of the second electrode is 0.1 V or more.

20. (canceled)

21. A sensing method comprising detecting that a particle having a size of 100 μm or less and containing an ionized molecule comes into contact with the first electrode and the second electrode, in a device including the first electrode and the second electrode, in which the first electrode and the second electrode are not electrically connected,
a shortest distance between the first electrode and the second electrode is 0.001 μm or more and 100 μm or less, and
an absolute value of a difference between a standard electrode potential of the first electrode and a standard electrode potential of the second electrode is 0.1 V or more.

22-24. (canceled)

25. A power generation method comprising generating power by bringing a particle having a size of 100 μm or less and containing an ionized molecule into contact with a first electrode and a second electrode, in a device including the first electrode and the second electrode, in which the first electrode and the second electrode are not electrically connected,
the shortest distance between the first electrode and the second electrode is 0.001 μm or more and 100 μm or less, and
an absolute value of a difference between a standard electrode potential of the first electrode and a standard electrode potential of the second electrode is 0.1 V or more.

26. (canceled)
